(19) United States
(12) Patent Application Publication
(10) Pub. No.: US 2004/0002141 A1
(43) Pub. Date: Jan. 1, 2004

(54) Methods and compositions for propagating vectors containing toxic cDNAs and ion channel assay systems

(76) Inventors: Martin Chalfie, New York, NY (US); Dattananda S. Chelur, New York, NY (US); Glen G. Ernstrom, New York, NY (US); C. Andrea Yao, New Haven, CT (US); Miriam B. Goodman, Palo Alto, CA (US)

Correspondence Address: BAKER & BOTTS, 30 ROCKEFELLER PLAZA, NEW YORK, NY, 10112

(21) Appl. No.: 10/338,324

(22) Filed: Jan. 8, 2003

Related U.S. Application Data
(60) Provisional application No. 60/348,077, filed on Jan. 10, 2002.
(60) Provisional application No. 60/357,609, filed on Feb. 15, 2002.
(60) Provisional application No. 60/360,092, filed on Feb. 26, 2002.
(60) Provisional application No. 60/364,569, filed on Mar. 14, 2002.
(60) Provisional application No. 60/390,835, filed on Jun. 20, 2002.

(51) Int. Cl. C12P 19/34; C12N 1/21; C12N 15/74; C12N 15/70

(52) U.S. Cl. 435/91.1; 435/252.3; 435/252.33; 435/320.1; 435/472; 435/488

(57) ABSTRACT

The present invention relates to methods and compositions which enable the propagation of vectors containing cDNAs whose presence has hitherto been toxic to conventional bacterial strains. It is based, at least in part, on the discovery that a bacterial strain having an insertional mutation in the malT gene of Escherichia coli tolerated the propagation of a mec-4 cDNA-containing plasmid which was toxic to other bacterial strains. The methods and compositions of the invention may be particularly useful in the propagation of cDNAs encoding membrane proteins. The present invention also provides for ion channel assay systems comprising MEC-2, MEC-4, MEC-10 or variants thereof.

### CROSS REFERENCE TO RELATED APPLICATIONS

This application claims priority to U.S. Provisional Patent Application Ser. No. 60/348,077, filed Jan. 10, 2002, to U.S. Provisional Patent Application Ser. No. 60/357,609, filed Feb. 15, 2002, to U.S. Provisional Patent Application Ser. No. 60/360,092, filed Feb. 26, 2002, to U.S. Provisional Patent Application Ser. No. 60/364,569, filed Mar. 14, 2002, and to U.S. Provisional Patent Application Ser. No. 60/390,835, filed Jun. 20, 2002, the entire contents of which are incorporated herein by reference.

### GRANT SUPPORT

The subject matter of this provisional specification was developed at least in part using funds provided by National Institutes of Health Grant No. GM 30997, so that the United States Government has certain rights herein.

### 1. INTRODUCTION

The present invention relates to methods and compositions which enable the propagation of vectors containing cDNAs whose presence has hitherto been toxic to conventional bacterial strains. It is based, at least in part, on the discovery that a bacterial strain having an insertional mutation in the malT gene of Escherichia coli tolerated the propagation of a mec-4 cDNA-containing plasmid which was toxic to other bacterial strains. The methods and compositions of the invention may be particularly useful in the propagation of cDNAs encoding membrane proteins. In another aspect, the present invention provides for ion channel assay systems comprising MEC-2, human stomatin, MEC-4 and/or MEC-10.

### 2. BACKGROUND OF THE INVENTION

Successful characterization of a cDNA depends upon the ability of that cDNA to be duplicated so as to produce sufficient amounts for further study. Because this duplication often involves transformation of a vector such as a plasmid containing this cDNA into a bacterial host, sufficient amounts of cDNA may be difficult or impossible to prepare if the presence of this vector is toxic to its host cell. A selection pressure to delete or mutate the toxic vector is created, which can lead to aberrant and misleading findings.

Standard protocols for preparing a cDNA library include preparing cDNA from a diverse mRNA population, inserting the resulting cDNAs into vectors, and transforming the cDNA-containing vectors into a culture of a bacterial host, usually Escherichia coli. The resulting population of transformed bacteria are intended to serve as a resource for retrieving cDNAs representative of the mRNA population. A vector containing a cDNA that is toxic to its bacterial host will result in that cDNA being underrepresented in the library. It is therefore desirable to develop methods and means which will permit the successful propagation of toxic vectors. Because membrane proteins may be underrepresented in cloning protocols, surmounting the problem of vector toxicity may increase the efficiency of cloning and characterizing membrane proteins.

Several strategies have been developed to solve this problem, some of which modify either the vector or its incorporated cDNA. For example, Worthington Biochemical Corp. of Lakewood, N.J. markets a plasmid, pT7-7, which places the cDNA under the control of the T7 promoter, which is not recognized by E. coli RNA polymerase, leading to low levels of expression of the cDNA (see Tabor and Richardson, Proc Natl Acad Sci USA February 1985; 82:1074-1078). Alternatively, Donnelly et al. (Protein Expr Purif August 2001; 22:422-9) describe the creation of an E. coli co-chaperone fusion protein that was better tolerated by host cells than the wild-type protein.

There are also bacterial strains commercially available intended to address the problem of vector toxicity. For example, Stratagene of La Jolla, Calif. markets “ABLE®” Competent Cells which, according to the company website, “reduce the copy number of common cloning vectors, enhancing the probability that a toxic clone will be propagated.” However, in an observation that led to the present invention, a plasmid containing an expressible form of the cDNA encoding the Caenorhabditis elegans mec-4 gene could not be successfully propagated in the ABLE® strains.

### 3. SUMMARY OF THE INVENTION

The present invention provides for methods and compositions which permit the propagation of otherwise toxic vectors in bacteria. The present invention is based, at least in part, on the discovery that a mutated strain of E. coli was able to tolerate propagation of a plasmid containing the cDNA for mec-4, which could not be propagated in commercially available E. coli strains. One particular E. coli strain, named SMC4, was found to be particularly efficient for propagating the mec-4-containing plasmid, but at least one other strain obtained by the mutagenesis and selection procedure was also found to be superior to commercial strains. When the mutations were characterized, it was found that the two toxic-vector-tolerant strains carried mutations in the malT locus, indicating that this locus is important in creating tolerance. However, one other bacterial strain containing a mutation in the malT locus did not support the growth of otherwise toxic vectors, indicating that other loci can impart resistance to toxic vectors.

Accordingly, in one embodiment, the present invention provides for a bacterial strain that propagates a toxic vector. In a particular embodiment, the invention provides for a bacterial strain that carries a mutation in the malT locus, and propagates a toxic vector. In a further embodiment, the bacterial strain carries a mutation in the malT locus and a second mutation at a locus other than the malT locus. In a further embodiment, the bacterial strain carries at least one other mutation at a locus other than the malT locus.

In another embodiment, the present invention provides for a method of producing a toxic-vector-tolerant bacterial strain comprising creating a mutation in wild-type bacteria, transforming the mutated bacterial strain with a toxic vector, and screening for the ability to propagate the vector. In a specific embodiment, a mutation is created in the malT locus. In a further embodiment, a mutation is created in the malT locus and a second mutation created at a locus other than the malT locus. In a further embodiment, the bacterial strain carries at least one other mutation at a locus other than the malT locus.

The successful propagation of vectors encoding toxic cDNAs has led to the expression of MEC-4 and MEC-10 in Xenopus laevis oocytes, and the discovery that the co-expression of mutant (“d” forms) of these proteins (MEC-4d and MEC-10d) produced a constitutively active, amiloride-sensitive ion channel. Additionally, MEC-2 was found to coactivate MEC-4/MEC-10 and, to an even greater extent, MEC-4d/MEC-10d, and MEC-4d expressed alone produced an ion channel.

Accordingly, in further embodiments, the present invention provides for compositions comprising homomeric or heteromeric complexes of wild-type or mutant MEC-2, human stomatin, MEC-4, and/or MEC-10, methods of preparing such compositions, and screening assays using the complexes for identifying ion channel modulating agents. In specific embodiments, human stomatin, or a variant thereof, may be substituted for MEC-2.

### 4. BRIEF DESCRIPTION OF THE FIGURES

FIG. 1A-H. MEC-4d, MEC-10d, and MEC-2 produce amiloride-sensitive currents. A, E—Voltage-dependence of amiloride difference currents. A. MEC-4d/MEC-10d (solid line), in these experiments MEC-4d and MEC-10d alone produced no amiloride-sensitive current (but see text, as in other experiments MEC-4d was observed to produce such a current). E. MEC-4d, MEC-10d, and MEC-2 in oocytes cultured with (solid line) and without (dotted line) amiloride. B, F—Time-dependence of currents evoked by voltage pulses between −100 and +35 mV (15 mV increments). Zero current (arrow). C, D, G, H—Membrane current (at −85 mV) and voltage in presence (+) and absence (−) of 300 μM amiloride. C, D. MEC-4d/MEC-10d (n=23). G, H. MEC-4d/MEC-10d and MEC-2 (n=9). V_{hold}=−60 mV; cells cultured with 300 μM amiloride, except as indicated. Vₘ=membrane potential. V_{hold}=holding potential. Iₘ=membrane conductance.

FIG. 2A-E. Functional interactions of MEC-4, MEC-10, and MEC-2. A, C—Amiloride-sensitive current amplitude (Measured at −85 mV in 3-88 cells cultured with and without amiloride). A. Wild-type and ‘d’ forms. D. With MEC-2. B. Surface expression of Myc::MEC-4d and MEC-10d::EGFP. D. Amiloride dose-response curves (at −60 mV) for MEC-4d, MEC-10d and MEC-2 (Kᵢ′=0.40 μM, n=21, filled) and MEC-4d and MEC-2 (Kᵢ′=3.2 μM, n=16, open). E. Voltage-dependence of amiloride blockade. The smooth line is a fit using a Woodhull model (Woodhull, 1973, J Gen Physiol 61:687-708) (δ=0.52 and 0.63 in the presence and absence of MEC-10d). EGFP=Enhanced green fluorescent protein).

FIG. 3A-C. MEC-2 interacts with MEC-4d and MEC-10d without altering surface expression. A. Co-immunoprecipitation of Myc::MEC-4d and MEC-10d::EGFP fusion proteins by antibodies against MEC-2. Five and one oocyte equivalent(s) were loaded in the IP and input lanes, respectively. B. Confocal images of live oocytes expressing MEC-4d and MEC-10d::EGFP in the presence (top) and absence (middle) of MEC-2. EGFP fluorescence is diffuse (bottom). C. Effect of MEC-2 on surface expression of MEC-4d (left) and MEC-10d (right). Each lane represents surface protein from 30-45 oocyte equivalents.

FIG. 4A-C. Three domains are needed for full MEC-2 function. A. Activity of truncated MEC-2 and human stomatin. Amiloride-sensitive current (at −85 mV in 8-32 cells) produced by co-expression with MEC-4d (bottom axis) and compared to full-length MEC-2 (top axis, % control). Dominant-negative effect of MEC-2(114-363) on: B. current amplitude (*P<0.01) and C. amiloride sensitivity. Normalized dose-response curves were obtained at −60 mV (left panel, n=4-16); Kᵢ′=0.93 and 3.2 μM with (filled) and without (open) MEC-2(114-363), respectively. Voltage-dependence of Kᵢ′ (right panel) with (filled, δ=0.68) and without (open, δ=0.63) MEC-2(114-363).

### 5. DETAILED DESCRIPTION OF THE INVENTION

In one aspect, the present invention relates to methods and compositions for propagating toxic vectors in bacteria. In this context, vectors can include plasmids, cosmids, bacterial artificial chromosomes (BACs), phagemids, bacteriophages, or any other vectors suitable for the propagation of DNA in bacterial hosts. Toxic vectors are vectors comprising sequences encoding toxic polypeptides such as, but not limited to, MEC-4, MEC-10, DEG-3, degenerin proteins, polypeptides demonstrating homology to a DEG/ENaC protein, transient receptor protein (TRP) ion channel proteins, TRP-related channel proteins, nucleoporin, brain sodium channel 1 (BNC1), and variants thereof.

In one embodiment, the present invention provides a method for generating toxic-vector-tolerant bacterial strains comprising mutagenizing a population of bacteria, transforming a mutagenized bacterial strain with a toxic vector, and screening for strength of colony formation. In a further embodiment, inverse PCR is performed to identify the region of the bacterial genome that has been mutagenized in the toxic-vector-tolerant strain.

Disruption of the malT gene was observed in two of the three strains characterized to date. The sequence of the malT gene in E. coli strain K-12 is available in GenBank at Accession Number M13585. The gene sequence may vary slightly between strains. Disruption of the malT gene may be detected by screening the transformants by selection methods for loss of ability to rely on maltose as a sole energy source, or by antibody-mediated screening, or by other methods known in the art, and may be confirmed by Southern blotting and/or amplification and sequencing.

The present invention further provides for bacterial strains that carry a mutation in the malT gene or that carry a mutation in control elements of the malT gene. In particular embodiments, the bacteria are E. coli bacteria.

In specific, nonlimiting embodiments, the malT gene has a mutation, such as an insertion, deletion, or substitution, preferably an insertion, in the region from about nucleotide 1000-3000 of the malT gene, based on the observation that successful insertions were documented at positions 1090 and 2603. In a preferred specific nonlimiting embodiment, the bacterial strain is SMC4, as deposited on Feb. 15, 2002 with the American Type Culture Collection (ATCC) located at 10801 University Boulevard, Manassas, Va. 20110-2209, and assigned accession number PTA-4084.

The bacterial strain can be derived from any bacteria including, but not limited to, bacteria from the family Acetobacteraceae, Acholeplasmataceae, Achromatiaceae, Acidimicrobiaceae, Acidothermaceae, Actinomycetaceae, Actinoplanaceae, Actinosynnemataceae, Aeromonadaceae, Alcaligenaceae, Alteromonadaceae, Anaeroplasmataceae, Anaplasmataceae, Aquificaceae, Archaeoglobaceae, Archangiaceae, Azotobacteraceae, Bacillaceae, Bacteroidaceae, Bartonellaceae, Beggiatoaceae, Bifidobacteriaceae, Bogoriellaceae, Branhamaceae, Brevibacteriaceae, Brucellaceae, Campylobacteraceae, Cardiobacteriaceae, Caryophanaceae, Caulobacteraceae, Cellulomonadaceae, Chlamydiaceae, Chlorobiaceae, Chromatiaceae, Chrysiogenaceae, Clostridiaceae, Comamonadaceae, Coriobacteriaceae, Corynebacteriaceae, Crenotrichaceae, Cystobacteraceae, Cytophagaceae, Deferribacteraceae, Deinococcaceae, Dermabacteraceae, Dermacoccaceae, Dermatophilaceae, Desulfurococcaceae, Dietziaceae, Ectothiorhodospiraceae, Ehrlichiaceae, Enterobacteraceae, Enterobacteriaceae, Enterobacteriaceae, Entomoplasmataceae, Ferroplasmaceae, Flavobacteriaceae, Frankiaceae, Gallionellaceae, Geodermatophilaceae, Glycomycetaceae, Gordoniaceae, Halanaerobiaceae, Haloanaerobiaceae, Halobacteriaceae, Halobacteroidaceae, Halomonadaceae, Hyphomicrobiaceae, Intrasporangiaceae, Jonesiaceae, Lactobacillaceae, Legionellaceae, Leptospiraceae, Leucotrichaceae, Lysobacteraceae, Methanobacteriaceae, Methanocaldococcaceae, Methanococcaceae, Methanocorpusculaceae, Methanomicrobiaceae, Methanoplanaceae, Methanopyraceae, Methanosaetaceae, Methanosarcinaceae, Methanospirillaceae, Methanothermaceae, Methylococcaceae, Microbacteriaceae, Micrococcaceae, Micromonosporaceae, Microsphaeraceae, Moraxellaceae, Mycobacteriaceae, Mycoplasmataceae, Myxococcaceae, Neisseriaceae, Nevskiaceae, Nitrobacteraceae, Nocardiaceae, Nocardioidaceae, Nocardiopsaceae, Oleiphilaceae, Oscillochloridaceae, Oscillospiraceae, Parachlamydiaceae, Pasteurellaceae, Pasteuriaceae, Peptococcaceae, Picrophilaceae, Planctomycetaceac, Planococcaceae, Polyangiaceae, Prochloraceae, Prochlorotrichaceae, Promicromonosporaceae, Propionibacteriaceae, Pseudomonadaceae, Pseudonocardiaceae, Pyrodictiaceae, Rarobacteraceae, Rhizobiaceae, Rhodospirillaceae, Rickettsiaceae Pinkerton, Rubrobacteraceae, Sanguibacteraceae, Simkaniaceae, Simonsiellaceae, Sphaerobacteraceae, Sphingobacteriaceae, Sphingomonadaceae, Spirillaceae, Spirochaetaceae, Spiroplasmataceae, Spirosomaceae, Sporichthyaceae, Streptococcaceae, Streptomycetaceae, Streptosporangiaceae, Succinivibrionaceae, Sulfolobaceae, Syntrophomonadaceae, Thermaceae, Thermococcaceae, Thermodesulfobacteriaceae, Thermofilaceae, Thermomicrobiaceae, Thermomonosporaceae, Thermoplasmataceae, Thermoproteaceae, Thermotogaceae, Thiocapsaceae, Treponemataceae, Tsukamurellaceae, Veillonellaceae, Verrucomicrobiaceae, Vibrionaceae, Vitreoscillaceae, or Waddliaceae.

In another embodiment, the present invention provides methods for the propagation of cDNAs encoding membrane proteins such as, but not limited to, the MEC proteins described herein, other DEG/ENaC proteins, other ion channel proteins, and receptor proteins. Non-limiting examples of such membrane proteins include but are not limited to UNC-1, UNC-8, DRASIC (Benson et al., 2002, Proc Natl Acad Sci USA. 99:2338-2343) and BNaClα (also known as ASIC2a and BNC1; Price, 2000, Nature 407:1007-1011). These methods of the invention comprise incorporating a cDNA encoding a membrane protein into a suitable vector and introducing the vector into a bacterial strain having tolerance to a toxic vector.

After identifying a bacterial strain that has a mutation in the malT locus, the ability of that strain to tolerate growth of a toxic “test” vector can be confirmed. For example, the size of colonies of a mutant malT bacterial strain transformed with either a toxic or a non-toxic vector can be compared, and may desirably be further compared to colonies of a similarly-transformed bacterial strain lacking the malT mutation. A smaller colony size in the wild-type compared to the malT mutant strains transformed with toxic vector indicates that the mutant is a toxic-vector-tolerant strain. As an example, a toxic “test” vector may contain the mec-4 gene.

Strains of bacteria having enhanced tolerance to toxic vectors may be obtained by subjecting a strain of bacteria having a mutation in malT to further mutagenesis, and screening the resulting bacteria for ability to support the propagation of a toxic vector.

In other embodiments, the present invention provides for bacterial strains with increased expression of malT that may favor vector copy number.

The successful propagation of cDNA encoding mec-4 led to the expression of MEC-4 and MEC-10 in Xenopus oocytes, and the discovery that the co-expression of the corresponding mutant (“d”) forms, MEC-4d and MEC-10d, produced a constitutively active, amiloride-sensitive ion channel. It was further discovered that MEC-2, a stomatin-like protein involved in touch sensitivity, increased the activity of the “d” mutant channels and allowed currents to be detected with wild-type MEC-4 and MEC-10. These findings demonstrate that MEC-2 regulates MEC-4/MEC-10 ion channels and indicates that similar ion channels may be formed by stomatin-like proteins and/or other DEG/ENaC proteins (see e.g., Bianchi and Driscoll, 2002, Neuron 34:337-340; Wood and Baker, 2001, Curr Opin Pharmacol 1:17-21; Mano and Driscoll, 1999, Bioessays 21:568-578) both in vertebrates and invertebrates. Such ion channels have been linked to mechanosensory responses. It has further been discovered that MEC-4d expressed in the absence of any of the aforelisted MEC proteins produced ion channels in Xenopus oocytes.

Accordingly, in various embodiments, the present invention provides for compositions comprising protein complexes comprising heteromers (multimers of more than one protein species) or homomers (multimers of one protein species) of MEC-2, human stomatin, MEC-4, MEC-10, or variants (i.e. mutants) of any of these proteins. A “complex” is defined herein as a multimer of the same or different proteins. For example, a complex may comprise one or more homodimer (one species of protein, e.g., MEC-4d₂), one or more heterodimer (two species of protein, e.g., MEC-4d/MEC-10d), one or more homotrimer (one species of protein), one or more heterotrimer (three species of protein, e.g., MEC-2/MEC-4d/MEC-10d or MEC-2/MEC-4/MEC-10 or MEC-2/MEC-4d/MEC-10 or MEC-2/MEC-4/MEC-10d), or combinations thereof to form larger multimers. In specific non-limiting embodiments, the present invention provides for complexes comprising heteromers of MEC-4d and MEC-10d and, in preferred embodiments, for heteromers of MEC-2, MEC-4d and MEC-10d. In other specific non-limiting embodiments, the present invention provides for homomers of variants of MEC-4, particularly MEC-4d. Of note, heteromers consisting essentially of MEC-2 and MEC-10 or MEC-10d, or of stomatin and MEC-10 or MEC-10d, have not been observed to produce ion channels.

The variants mentioned herein are collectively referred to as “MEC-variants” for proteins and “mec-variants” for nucleic acids.

In another embodiment, the present invention provides for ion channels that are modulated by MEC-2 or a MEC-2 variant (e.g., stomatin). In a specific embodiment, MEC-2, or a variant thereof, stimulates an amiloride-sensitive current. In a further embodiment, MEC-2 or a variant thereof contacts the ion channel to activate or enhance an amiloride-sensitive current.

In one embodiment, the mec-variant has 60, 65, 70, 75, 80, 85, 90, 95, 96, 97, 98, or 99 percent overall identity in the nucleotide sequence compared to the wild-type sequence. In another embodiment, the mec-variant has 60, 65, 70, 75, 80, 85, 90, 95, 96, 97, 98, or 99 percent identity in the nucleotide sequence of a domain compared to the corresponding domain of the wild-type sequence. In another embodiment, the MEC-variant has 60, 65, 70, 75, 80, 85, 90, 95, 96, 97, 98, or 99 percent overall identity in the amino acid sequence compared to the wild-type sequence. In yet another embodiment, the MEC-variant has 60, 65, 70, 75, 80, 85, 90, 95, 96, 97, 98, or 99 percent identity in the amino acid sequence of a domain compared to the corresponding domain of the wild-type protein. In a specific embodiment, the amino acid sequence of a variant of MEC-2 is about 64% identical to the central domain (amino acids 114-363) of wild-type MEC-2. Non-identity may arise from deletion, insertion, or substitution of one or more nucleic acid or amino acid residues.

In another embodiment, the variant has a post-translational modification not normally present in the wild-type polypeptide.

In compositions of the invention, the MEC proteins are provided in a context other than their naturally occurring cellular environment, for example, but not by limitation, in vitro or in an a heterologous expression system such as Xenopus laevis oocytes, CHO cells, HEK 293 cells, etc.

Another aspect of the present invention provides for methods of preparing the compositions of the invention. Such methods include, but are not limited, co-expression of complex constituents.

Another aspect of the present invention provides for screening assays using the compositions of the invention. Accordingly, in one embodiment, the present invention provides for methods of identifying ion-channel-modulating agents comprising contacting an ion channel of the invention with a test compound and measuring modulating effects on ion channel function.

In another embodiment, the present invention provides for methods of identifying agents that modulate a mechanosensory response comprising contacting an ion channel of the invention with a test compound and measuring modulating effects on an index of a mechanosensory response. Possible indices include but are not limited to a change in membrane potential, ion current, and a change in conformation of cytostructural elements.

In particular embodiments, the present invention provides for a method of identifying an agent that binds to an ion channel-containing heteromeric complex of MEC-2, human stomatin, MEC-4 and/or MEC-10 (or variants thereof), and does not bind to monomers or homomers or heteromers of the constituent proteins which lack ion channel activity. In another embodiment, the invention provides for a method of identifying an agent that binds to a homomeric complex of MEC-4d, and does not bind to a MEC-4 or MEC-4d monomer. The skilled artisan can readily appreciate the various combinations of MEC-2, human stomatin, MEC-4 and/or MEC-10 useful for above screening assays. The ability of an agent to bind to a protein complex that contains an ion channel, together with an inability to bind to the corresponding proteins in a configuration which does not have ion channel activity, indicates that the agent may be useful in modulating ion channel function.

### 6. EXAMPLE

Touch sensitivity in animals relies on nerve endings in the skin that convert mechanical force into electrical signals. In the nematode Caenorhabditis elegans, gentle touch to the body wall is sensed by six mechanosensory neurons (Chalfie and Sulston, 1981, Dev Biol 82:358-370) that express two amiloride-sensitive Na⁺ channel proteins (DEG/ENaC). These proteins, MEC-4 and MEC-10, are required for touch sensation and can mutate to cause neuronal degeneration (Driscoll and Chalfie, 1991, Nature 349:588-593; Huang and Chalfie, 1994, Nature 367:467-470). Data presented herein demonstrate that these mutant (i.e., ‘d’ forms) of MEC-4 and MEC-10 produce a constitutively-active, amiloride-sensitive ionic current when co-expressed in Xenopus oocytes. MEC-2, a stomatin-related protein needed for touch sensitivity (Huang et al., 1995, Nature 378:292-295), increased the activity of mutant channels ˜40-fold and allowed currents to be detected with wild-type MEC-4 and MEC-10. Whereas neither the central stomatin-like domain of MEC-2 nor human stomatin retained the activity of full-length MEC-2, both produced amiloride-sensitive currents with MEC-4d. Our findings demonstrate that MEC-2 regulates MEC-4/MEC-10 ion channels and indicate that similar ion channels may be formed in both vertebrates and invertebrates by stomatin-like proteins and DEG/ENaC proteins that are co-expressed (Tavernarakis et al., 1997, Neuron 18:107-119; Mannsfeldt et al., 1999, Mol Cell Neurosci 13:391-404; Fricke et al., 2000, Cell Tissue Res 299:327-334; Sedensky et al., 2001, Am J Physiol Cell Physiol 280:C1340-1348). These channels may mediate mechanosensory responses.

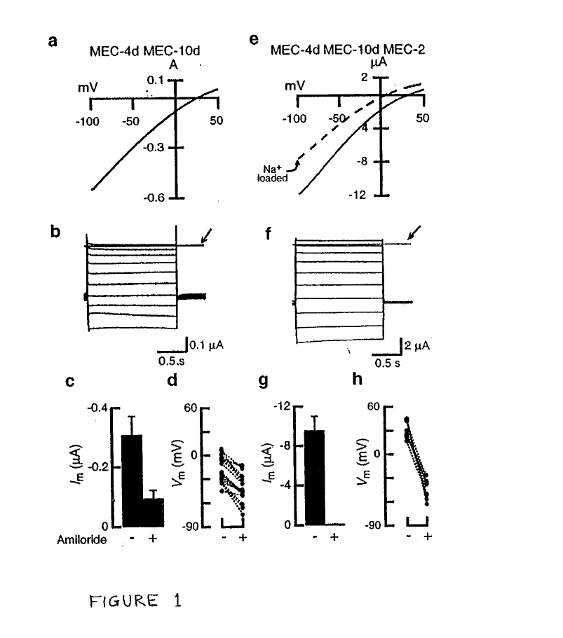

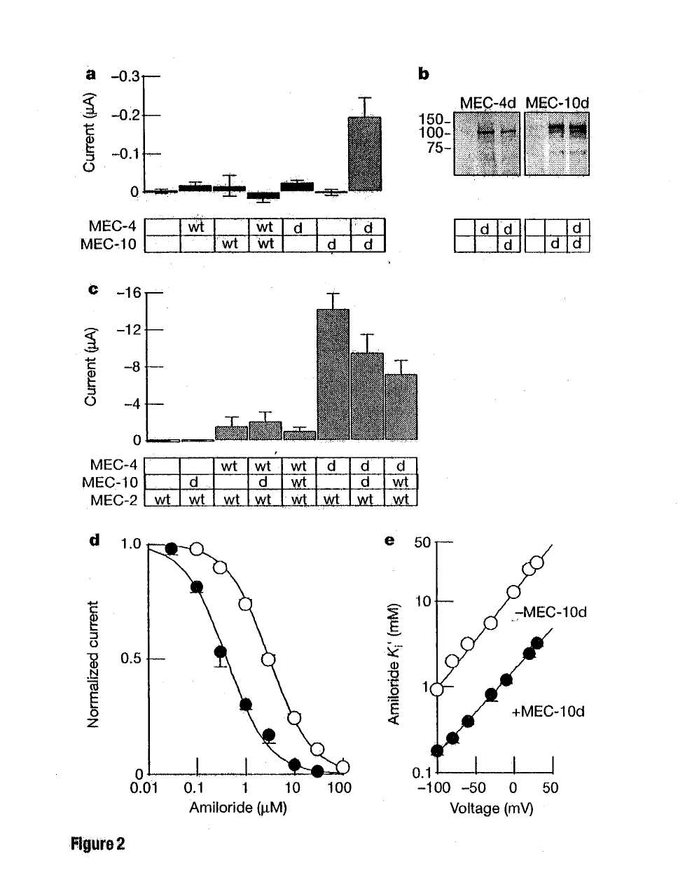

MEC-4 and MEC-10, which are 53% identical, function non-redundantly in mechanosensation (Chalfie and Sulston, 1981, Dev Biol 82:358-370; Chalfie and Au, 1989, Science 243:1027-1033). As found for related DEG/ENaC channels from worms (García-Añoveros et al., 1998, Neuron 20:1231-1241), flies (Adams et al., 1998, J Cell Biol 140:143-152), and humans (Waldmann et al., 1996, J Biol Chem 271:10433-10436), no amiloride-sensitive current was detected in oocytes expressing one or both wild-type proteins. Originally, the data demonstrated that the ‘d’ forms produced amiloride-sensitive currents only when expressed together (FIG. 1A, 2A), even though both proteins were present in the plasma membrane when expressed alone (FIG. 2B). The observed requirement for both proteins was not due to a change in the amount of surface protein; the ratio of MEC-4d vs. MEC-4d/MEC-10d was 0.98 (n=2) and for MEC-10d vs. MEC-4d/MEC-10d, it was 1.04 (n=2). The current produced by MEC-4d/MEC-10d displayed a mild inward-rectification and lacked any obvious time-dependent component (FIG. 1B). These results suggested that gain-of-function mutations that cause cell death in vivo and that activate other DEG/ENaC channels (Waldmann et al., 1996, J Biol Chem 271:10433-10436; Adams et al., 1998, J Cell Biol 140:143-152; García-Añoveros et al., 1998, Neuron 20:1231-1241) also activate MEC-4/MEC-10. Consistent with this idea, MEC-4d/MEC-10d elevated the resting membrane potential (Vₘ), and made Vm sensitive to amiloride (FIG. 1D).

On examining additional amiloride-treated oocytes, it was found that injection of Xenopus oocytes with MEC-4d capped RNA (“cRNA”) alone resulted in a statistically significant amiloride-sensitive current at −85 mV (P<0.0001). Cells expressing MEC-4d produced amiloride-sensitive currents of −0.22±0.03 μA (n=53, range: −0.007 to −1.15 μA), compared to −0.005±0.006 μA (n=19, range: −0.06 to +0.06 μA) for water-injected controls. Although these currents are similar in average size, voltage-dependence and time-dependence to those measured in cells co-expressing MEC-4d and MEC-10d, these results do not alter the conclusion that MEC-4 and MEC-10 form heterodimeric channels, since MEC-10d increases the apparent affinity for amiloride of the MEC-4d/MEC-2 channel (see below). The finding that MEC-4d cRNA alone produces an amiloride-sensitive current indicates, however, that MEC-4 forms homomeric channels when expressed alone.

The MEC-4d/MEC-10d current was carried by Na⁺ ions, since it reversed polarity at 15±3 mV (n=18) and was essentially eliminated by substituting K⁺ for Na⁺ in the external saline. Like αβγENaC (Canessa et al., 1994, Nature 367:463-467), this current was more permeable to Li⁺ than Na⁺ (P_{Li}/P_{Na}=3.1±0.5, n=6). The permeability of the MEC-4d/MEC-10d current differs from that of the C. elegans DEG/ENaC protein UNC-105d, which forms channels that are less permeable to lithium ions and more permeable to potassium ions (García-Añoveros et al., 1998, Neuron 20:1231-1241). Similarly, the apparent amiloride inhibition constant (Ki′), which was 0.12±0.03 μM (n=6) at −100 mV, was similar to that of αβγENaC (Canessa et al., 1994, Nature 367:463-467), but significantly smaller than that of UNC-105d (García-Añoveros et al., 1998, Neuron 20:1231-1241). Thus, the properties of MEC-4d/MEC-10d currents more closely resemble those of the mammalian ENaC channel than the C. elegans UNC-105 channel.

Genetic interactions suggest that MEC-2, which is expressed in all six touch cells (Huang et al., 1995, Nature 378:292-295), regulates MEC-4/MEC-10 ion channels (Huang and Chalfie, 1994, Nature 367:467-470). Functional interactions were tested by co-expressing MEC-2 with MEC-4d and MEC-10d in Xenopus oocytes (FIG. 1E-H). MEC-2, which had no effect on membrane current when expressed alone (FIG. 2C), increased the amplitude of amiloride-sensitive currents ˜40-fold but did not affect their voltage- or time-dependence (compare FIG. 1C with FIG. 1G). MEC-2 may regulate the ion permeation pathway, since it reduced the relative permeability for Li+ ions (P_{Li}/P_{Na}=1.44±0.07, n=16). Interactions between genes encoding UNC-1 (a stomatin-like protein) and UNC-8 (a DEG/ENaC protein; Rajaram et al., Genetics 153:1673-1682) suggest that this activity is shared by other stomatin-like proteins in C. elegans.

The sodium current produced by co-expressing MEC-4d, MEC-10d, and MEC-2 drives Vₘ toward the expected Nernst potential for Na⁺ ions, E_{Na} (Vₘ=+32±4 mV, n=9) in oocytes cultured in the presence of 300 μM amiloride. Oocytes cultured without amiloride, by contrast, had resting potentials close to 0 mV (Vm=−1.34±1 mV, n=30). This observation is reminiscent of the “Na⁺ loading” effect described for αβγENaC channels (Canessa et al., 1994, Nature 367:463-467) and is reflected in a shift in the reversal potential of the amiloride-sensitive current (FIG. 1B; control Eᵣₑᵥ=32±1 mV, n=9 vs. Na⁺ loaded Eᵣₑᵥ=4±2 mV, n=27). Thus, culturing oocytes without amiloride drives E_{Na} close to 0 mV.

The amplification provided by MEC-2 allowed us to detect amiloride-sensitive currents in some oocytes (13 of 33 cells from 3 of 6 frogs) expressing wild type MEC-4 and MEC-10 (FIG. 2C), which produced no amiloride-sensitive current when expressed alone or together (FIG. 2A). This current was observed in the absence of any explicit mechanical stimulation. Current could not be induced in oocytes lacking a constitutive, amiloride-sensitive current by superfusion with salines having either reduced pH (5.2, n=3) or osmolarity (115 mOsm, n=16). This result implies that wild-type channels with MEC-2 may be partially open at rest (i.e., open probability, Pₒ>0). A similar situation may occur in vivo. Even a tiny resting Na⁺ current would depolarize touch cells, since they exhibit an unusually high input resistance (of at least 2 GΩ). In this case, a change in mechanical force could hyperpolarize touch cells by closing channels and/or depolarize them further by opening channels. Alternatively, other proteins important for touch cell function in vivo may decrease resting Pₒ. In particular, components of the touch cell extracellular matrix and/or cytoskeleton, which are important or required for touch sensitivity in vivo (Chalfie and Sulston, 1981, Dev Biol 82:358-370; Chalfie and Au, 1989, Science 243:1027-1033), may prevent the channel from opening at rest. The response to mechanical force would remove inhibition generated by interaction with specialized structures and allow channels to assume their resting Pₒ, resulting in depolarization. In this scenario, the channel would not be directly mechanically-gated, but would be mechanically sensitive by virtue of its interaction with other proteins in vivo.

MEC-4 and MEC-10 exhibit functional differences when co-expressed with MEC-2 in Xenopus oocytes. Specifically, introducing the ‘d’ mutation into MEC-4, but not MEC-10, significantly increased current amplitude (FIG. 2C), a difference that may account for the comparatively weak degeneration phenotype observed with mec-10d (Huang and Chalfie, 1994, Nature 367:467-470). It was determined that MEC-4, but not MEC-10, was both necessary and sufficient to produce amiloride-sensitive currents in the presence of MEC-2 (FIG. 2C). This result agrees with genetic studies showing that mec-4 is required for neuronal degeneration caused by mec-10d, but not vice versa (Huang and Chalfie, 1994, Nature 367:467-470), but raises the question of whether or not each protein forms distinct channels or a single heteromeric channel. As described above, the finding that MEC-4d cRNA alone produces an amiloride-sensitive current indicates that MEC-4 forms distinct channels.

Amiloride dose-response curves were determined to answer this question and found that adding MEC-10d reduced Ki′ without introducing a second class of binding sites (FIG. 2D). Scatchard plots were also consistent with the existence of a single class of binding sites in the presence and absence of MEC-10d. These observations indicate that MEC-4d and MEC-10d form a heteromeric channel. A single amiloride molecule may bind to each channel and inhibit current by lodging in the ion pore formed by MEC-4d and MEC-10d, as proposed for native ENaC channels (Palmer, 1985, J Membr Biol 87:191-199). Consistent with this idea, amiloride blockade was steeply voltage-dependent (FIG. 2E). Analysis of the relation between Ki′ and voltage revealed that amiloride penetrated at least 50% of the membrane electric field (FIG. 2E, δ=0.54-0.62). These values are similar to those measured for channels formed by UNC-105d (δ=0.65-0.68; García-Añoveros et al., 1998, Neuron 20:1231-1241), but greater than those of the αβγENaC channels (δ=0.15; McNicholas and Canessa, 1997, J Gen Physiol 109:681-692). Although the molecular basis for this difference is unknown, it could reflect differences in the accessibility of the amiloride binding site and/or its location with respect to the membrane electric field.

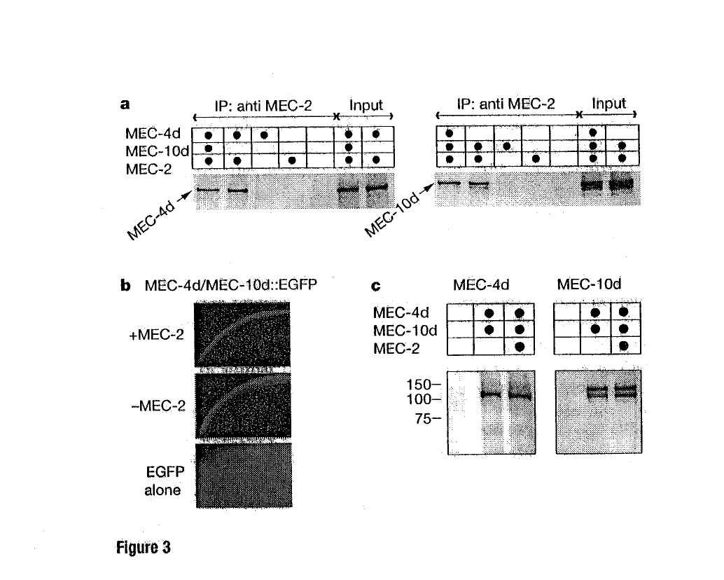

Antibodies against MEC-2 immunoprecipitated MEC-4d and MEC-10d (FIG. 3A) indicating that MEC-2 forms an ion channel complex with MEC-4 and MEC-10, a result consistent with genetic studies (Huang and Chalfie, 1994, Nature 367:467-470; Gu et al., 1996, Proc Natl Acad Sci USA 93:6577-6582). The interaction is pair-wise: MEC-2 immunoprecipitated MEC-4d in the absence of MEC-10d and vice versa. It is also specific, since MEC-2 failed to immunoprecipitate the endogenous membrane protein, β-integrin (Muller et al., Mech Dev 42:77-88). To test whether the increased current size was produced by an increase in the number of MEC-4d/MEC-10d channels that reach the plasma membrane, MEC-4d and MEC-10d were tagged. A MEC-10d::EGFP fusion protein was visible near the plasma membrane of live oocytes (FIG. 3B) and produced amiloride-sensitive currents when co-expressed with MEC-4d and MEC-2 (see Methods section below). MEC-10d::EGFP localization was not obviously affected by omitting MEC-2. MEC-2 also did not affect the amount of either MEC-4d or MEC-10d available for biotinylation at the surface (FIG. 3C); the ratio of MEC-4d with and without MEC-2 was 1.2±0.4 (n=5), and the ratio of MEC-10d with and without MEC-2 was 0.9±0.3 (n=3). MEC-2 is, therefore, unlikely to increase channel number and likely acts by regulating single channel conductance, open probability, and/or mean open time.

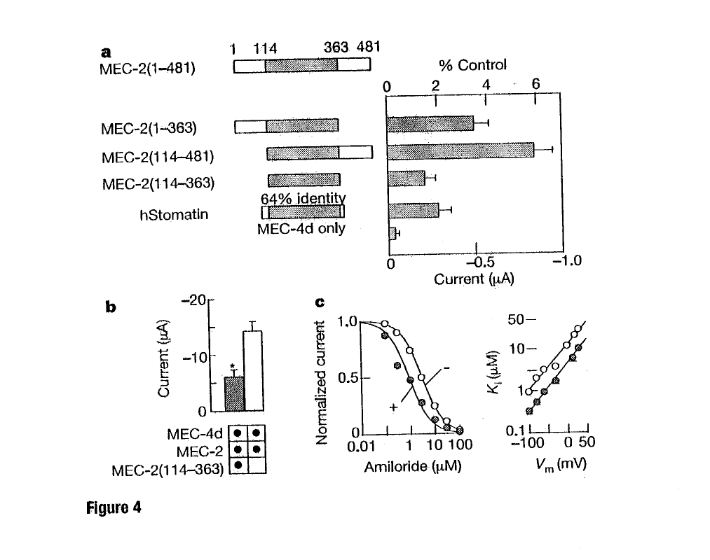

The central domain of MEC-2 (amino acids 114-363) is 64% identical to stomatin, a human protein implicated in the regulation of ion flux in red blood cells (Lande et al., J Clin Invest 70:1273-1280). Fifty-four alleles of mec-2 were identified in genetic screens for touch-insensitive mutants (Chalfie and Sulston, 1981, Dev Biol 82:358-370; Chalfie and Au, 1989, Science 243:1027-1033). More than half of these are missense mutations that map to this central, stomatin-like domain (Huang et al., 1995, Nature 378:292-295), indicating that this domain is especially important for the function of MEC-2. To determine whether stomatin and MEC-2(114-363) function similarly, it was co-expressed with the ‘d’ form of MEC-4. MEC-2(114-363) produced comparatively small amiloride-sensitive currents (FIG. 4B), indicating that the central domain retains the ability to generate amiloride-sensitive currents with MEC-4d. Stomatin, which had no effect by itself (n=13), produced amiloride-sensitive currents of a similar size when co-expressed with MEC-4d (FIG. 4A). These findings establish that MEC-2(114-363) and stomatin share the ability to regulate a DEG/ENaC channel and provide the first demonstration that stomatin-like proteins regulate ion channels.

The stomatin-like domain of MEC-2(114-363) reduces current amplitude in a dominant-negative fashion when co-expressed with full-length MEC-2 (FIG. 4B). Human stomatin also produced a strong dominant-negative effect, reinforcing the functional similarity between the two proteins. Such interference indicates that MEC-2 forms multimers via the conserved central domain, which is also supported by interallelic complementation at mec-2 (Chalfie and Sulston, 1981, Dev Biol 82:358-370; Huang, 1995, Ph.D. Thesis, Columbia University) and by physical interactions between stomatin monomers (Snyers et al, 1998, J Biol Chem 273:17221-17226). MEC-2(114-363) also reduced amiloride Ki′, without introducing an additional class of binding sites or changing the voltage-dependence of blockade (FIG. 4C), a finding which suggests that while MEC-2 may regulate access to the amiloride binding site or contribute to its formation, it does not regulate the position of the binding site within the electrical field.

Both unique N-terminal and C-terminal regions of MEC-2, which are believed to be cytoplasmic (Huang and Chalfie, 1994, Nature 367:467-470), are needed for full activity of the protein in Xenopus oocytes. Whereas the addition of either terminal domain increased current amplitude to some extent, neither MEC-2(1-363) nor MEC-2(114-481) produced currents as large as those observed with the full-length protein (FIG. 4B). This reduction did not reflect a reduction in protein levels, since both truncated proteins were produced at approximately the same level as the full-length protein (as judged by Western blot analysis). Thus, all three domains contribute to the function of MEC-2.

Thus, the highly conserved, stomatin-like domain of MEC-2 likely provides an essential structural scaffold for interaction with DEG/ENaC proteins, with the lipids surrounding the channel, or both. Evidence for lipid association comes from the observation that stomatin is palmitoylated in vivo (Snyers et al., 1999, FEBS Lett 449:101-104) and associated with lipid rafts (Snyers et al., 1999, FEBS Lett 449:101-104; Salzer et al., 2001, Blood 97:1141-1143), sphingolipid- and cholesterol-rich microdomains in the plasma membrane. The predominant site of palmitoylation in stomatin (Snyers et al., 1999, FEBS Lett 449:101-104) is conserved in MEC-2 and such a covalent modification, if present, would anchor MEC-2 to the inner leaflet of the plasma membrane. Although MEC-2(114-363) acts in a dominant-negative fashion, the majority of the ability of MEC-2 to regulate ion channel function is explained by the action of the unique amino and carboxyl termini. The central stomatin-like domain may, therefore, bring these unique domains in close proximity to MEC-4 and MEC-10.

The reconstitution of channel activity in Xenopus oocytes establishes the biochemical function of MEC-4, MEC-10, and MEC-2 and is a first step toward understanding the function of other proteins implicated in C. elegans mechanosensation. The physical and functional interactions detailed can be applied to homologous proteins in vertebrates, and to determine any role in mechanosensation. A role for DEG/ENaC proteins in vertebrate mechanosensation comes from recent studies showing that BNaC1α(also known as ASIC2a and BNC1), is present in the somata and terminals of dorsal root ganglion (DRG) neurons that innervate mammalian skin (García-Añoveros et al., 2001, J Neurosci 21:2678-2686) and is needed for normal sensory responses in one class of sensory nerve fibers (Price et al., 2000, Nature 407:1007-1011). Stomatin may regulate the channel containing BNC1, since it is expressed in all DRG neurons (Mannsfeldt et al., 1999, Mol Cell Neurosci 13:391-404). Stomatin is also co-expressed with αβγENaC channels in trigeminal sensory neurons that sense whisker deflections in rats (Fricke et al., 2000, Cell Tissue Res 299:327-334) and may regulate these channels. Co-expression of human stomatin or MEC-2(114-363) with MEC-4d continues to produce a small increase in MEC-4d current (P<0.05), indicating that stomatin-like proteins share the common function of regulating DEG/ENaC ion channels. Such interactions would expand the combinatorial possibilities for channel activity beyond that previously imagined for DEG/ENaC proteins alone. The new combinations can be used to identify agents that bind to or modulate the ion channels, and can be used to identify agents that modulate the mechanosensory response.

### Methods

Expression Constructs. Wild-type cDNAs encoding full-length MEC-2 (Fricke et al., 2000, Cell Tissue Res 299:327-334), truncated MEC-2 proteins, MEC-4, and MEC-10 were subcloned into pGEM-HE or pSGEM with a Kozak sequence upstream of the initial codon. Plasmids encoding wild-type MEC-4 (TU#667) and MEC-10 (TU#668) were mutated in vitro to give plasmids encoding MEC-4d (TU#655) and MEC-10d (TU#656). These mutations reproduce the mec-4(e1611) (Driscoll and Chalfie, 1991, Nature 349:588-593) mutation and correspond to A713T and A673T in MEC-4 and MEC-10, respectively. Coding sequences were verified by automated DNA sequencing.

Bacterial Strain for Degenerin Plasmids. Plasmids containing full-length degenerin cDNAs are toxic to standard E. coli strains (Huang and Chalfie, 1994, Nature 367:467-470; Lai et al., 1996, J Cell Biol 133:1071-1081); transformants either form tiny colonies or carry mutant plasmids. An E. coli strain, SMC4 (ATCC Accession No. PTA-4084), was generated by randomly mutating E. coli NM554 with the mini-Tn10 cam transposon (Kleckner et al., Methods Enzymol 204:139-180), transforming with a mec-10 plasmid, and screening for normnal growth. SMC4 demonstrated normal growth with mec-4 and mec-10 plasmids and stable propagation of the mec-4 and mec-10 plasmids. Stable propagation was tested by showing that the plasmid caused NM554 and XL2 blue to give tiny colonies, curing the strain of the plasmid, and testing for growth of a mec-4 plasmid. TU#667, TU#668, TU#655, and TU#656 and their derivatives were propagated in SMC4.

Oocyte Expression & Electrophysiology. Capped RNAs (“cRNAs”) were synthesized (T7 mMESSAGE mMACHINE™ kit, Ambion, Austin, Tex.), purified, and quantified spectroscopically. Xenopus laevis oocytes were harvested and injected with 10 ng of each cRNA, except for oocytes co-expressing only MEC-4d and MEC-2(114-363), which were injected with 10 ng of the former and 20 ng of the latter. Oocytes were maintained in L-15 oocyte medium containing 100 μg/mL gentamicin (Cell & Molecular Technologies, Philipsburg, N.J.) at 16-18° C. Where indicated, 300 μM amiloride was added to the culture medium.

Membrane potential and current were measured 4-10 days after cRNA injection using a two-electrode voltage clamp (Warner OC-725C) at 22-25° C. Electrodes (0.3-2 MΩ) were filled with 3 M KCl and oocytes were superfused with saline containing (in mM): Na-gluconate (100), KCl (2), CaCl₂ (1), MgCl₂ (2), NaHEPES (10), pH 7.2. For low pH experiments, HEPES was replaced by MES. For hypo-osmotic experiments, saline was diluted to 100-110 mOsm. Current was similar in hypo-osmotic saline supplemented with sucrose. Ion selectivity experiments used salines containing (in mM): X⁺-gluconate (100), CaCl₂ (1), MgCl₂ (2) and X⁺ HEPES (10), pH 7.2, where X⁺ was Li⁺, Na⁺, K⁺, Cs⁺, or n-methyl-d-glucamine (NMG). Amiloride-difference currents were used to determine ion selectivity of MEC-4d/MEC-10d channels. All chemicals were obtained from Sigma (St. Louis, Mo.).

Analog signals were filtered at 200 Hz and sampled at 1-2 kHz (ITC-16, Instrutech, Great Neck, N.Y.); a 60 Hz notch filter was used to minimize line noise. Average values are reported as mean±S.E.M. Curves were fit by a nonlinear least-squares method (IgorPro 4.01, Wavemetrics, Oswego, Oreg.); the standard deviation measured at each point provided the weighting function. For dose-response relations, current was normalized to the total amiloride-sensitive current (measured as the difference in control and 300 μM amiloride salines). Relative permeabilities were calculated from the difference in reversal potential measured in solutions containing Na⁺ and each test ion using the Goldman-Hodgkin-Katz equation (Hille, 2001, In: Ion Channels of Excitable Membranes, Sinaur Associates, Inc., Sunderland, Mass.).

Surface Expression, Co-immunoprecipitation, & Western Blotting. Surface expression was assayed according to Chillaron et al.(Chillaron et al., 1997, J Biol Chem 272:9543-9549). Treatment groups were normalized to 30-40 oocyte equivalents/lane and subject to SDS-PAGE followed by western blotting. The C-terminus of MEC-10d was fused to EGFP (Clontech, Palo Alto, Calif.). When co-expressed with MEC-4d and MEC-2, this fusion protein generated amiloride sensitive currents [Iₐₘᵢₗ(−85 mV)=−5.3±1 μA, n=6] with a slightly elevated Ki′ for amiloride [Ki′(−60 mV)=1.0±0.2 μM, n=7]. Cytoplasmic EGFP was detected in the supernatant (internal) fraction, but not in the streptavidin precipitate. The N-terminus of MEC-4d was fused to Myc; Myc::MEC-4d was functional when co-expressed with MEC-10d and MEC-2 [Iₐₘᵢₗ(−85 mV)=−9.8±3 μA, n=3]. MEC-10d::EGFP and Myc::MEC-4d were detected either with HRP-conjugated antibodies against the epitope tags (Santa Cruz Biotechnology, Santa Cruz, Calif.) or with primary antibodies against the epitope tags (Zymed, South San Francisco, Calif.) and HRP-conjugated secondary antibodies. HRP was detected using chemiluminescence (ECL and ECLplus, Amersham Pharmacia Biotech, Piscataway, N.J.). Band density was measured from digitized films using NIH Image; intensity was corrected post hoc for variation in oocyte equivalents loaded.

Ion channel complexes were immunoprecipitated from oocyte homogenates with rabbit polyclonal antibodies raised against purified, bacterial MEC-2(145-481). Homogenates were prepared 5-6 days after cRNA injection using 10 μL of lysis buffer (20 mM Tris-HCl, pH 7.6, 100 mM NaCl, 2% NP-40) per oocyte. Yolk platelets were removed by low-speed centrifugation and the supernatant diluted with lysis buffer to a final concentration of 2-10 oocytes/mL. Immunocomplexes were precipitated by Protein A/G PLUS conjugated to agarose (Santa Cruz Biotechnology, Santa Cruz, Calif.), washed three times in lysis buffer, and analyzed by SDS-PAGE. Four to five oocyte equivalents were loaded per “IP” lane; one oocyte equivalent was loaded per input lane. Western blotting was essentially as described above. The specificity of the interaction was confirmed in two ways: (1) using anti-Myc and anti-EGFP antibodies conjugated to agarose to immunoprecipitate MEC-2 from the same sample, and (2) probing immuno-complexes for the presence of β-integrin with a monoclonal antibody (8C8, Developmental Studies Hybridoma Bank, University of Iowa, Iowa City, Iowa), an unrelated, Xenopus oocyte membrane protein (Muller et al., 1993, Mech Dev 42:77-88).

Various publications are cited herein which are hereby incorporated by reference in their entireties.

We claim:

1. A method for producing a bacterial strain which tolerates the propagation of a toxic vector, comprising exposing a bacterial culture to a mutagen and then identifying and isolating a bacterium which has developed a mutation in the malT gene, and culturing the isolated bacterium to produce the toxic-vector-tolerant bacterial strain.

2. The method of claim 1, where the bacterial strain is an Escherichia coli strain.

3. The method of claim 1, wherein the vector is selected from the group consisting of plasmids, cosmids, bacterial artificial chromosomes (BACs), phagemids, or bacteriophages.

4. A bacterial strain produced by the method of claim 1.

5. A bacterial strain that is tolerant to the propagation of a toxic vector and which carries a mutation in the malT gene.

6. The bacterial strain of claim 5, wherein the vector is selected from the group consisting of plasmids, cosmids, bacterial artificial chromosomes (BACs), phagemids, or bacteriophages.

7. The bacterial strain of claim 6 which is an Escherichia coli strain.

8. The bacterial strain of claim 6 which is SMC4, as deposited with the American Type Culture Collection and assigned Accession No. PTA-4084.

9. A method of propagating a toxic vector, comprising transforming a bacterium of the bacterial strain of claim 5 with the vector and then culturing the bacterium.

10. A method of propagating a toxic vector, comprising transforming a bacterium of the bacterial strain of claim 6 with the vector and then culturing the bacterium.

11. A method of propagating a toxic vector, comprising transforming a bacterium of the bacterial strain of claim 7 with the vector and then culturing the bacterium.

12. A method of propagating a toxic vector, comprising transforming a bacterium of the bacterial strain of claim 8 with the vector and then culturing the bacterium.

13. A composition comprising a complex selected from the group consisting of (i) a heteromeric complex comprising two or more proteins selected from the group consisting of MEC-2, human stomatin, MEC-4, MEC-10, and a MEC-variant, where the complex does not consist essentially of a combination of proteins selected from the group consisting of MEC-2 and MEC-10, MEC-2 and MEC-10d, human stomatin and MEC-10 and human stomatin and MEC-10d, and (ii) a homomeric complex of a protein selected from the group consisting of MEC-4 and a variant thereof.

14. The composition of claim 13 wherein said complex comprises an ion channel.

15. The composition of claim 13 wherein said complex comprises MEC-4d and MEC-10d.

16. The composition of claim 13 wherein said complex comprises a MEC-2 variant, MEC-4d and MEC-10d.

17. A method for identifying an ion-channel-modulating agent comprising (1) contacting an ion channel of claim 14 with a test compound; and (2) measuring modulating effects on ion channel function.

18. The method of claim 17 wherein said ion channel is comprised in a heteromeric complex of MEC-2, MEC-4, and MEC-10, or one or more variant thereof; wherein said agent binds to said heteromeric ion channel-forming complex; and wherein said agent does not bind to a heteromeric or homomeric complex of any one or more constituent protein which does not form an ion channel.

19. A method for identifying an agent that modulates a mechanosensory response comprising (1) contacting an ion channel of claim 14 with a test compound; and (2) measuring a modulating effect on an index of a mechanosensory response.

20. An assay system for identifying an agent that modulates an ion channel comprising the complex of claim 13.
